(19) United States
(12) Patent Application Publication
(10) Pub. No.: US 2016/0339202 A1
(43) Pub. Date: Nov. 24, 2016

(54) VENTILATORS AND VENTILATOR SYSTEMS

(71) Applicant: SMITHS MEDICAL INTERNATIONAL LIMITED, Ashford (GB)

(72) Inventors: Robert James Burchell, Hertfordshire (GB); David James Baker, Souillac (FR); Clive John Millward, Bedfordshire (GB); Mark Sinclair Varney, Bedfordshire (GB)

(73) Assignee: SMITHS MEDICAL INTERNATIONAL LIMITED, Ashford (GB)

(21) Appl. No.: 15/114,876

(22) PCT Filed: Jan. 24, 2015
(86) PCT No.: PCT/GB2015/000022

§ 371 Date: Jul. 28, 2016

(30) Foreign Application Priority Data
Feb. 6, 2014 (GB) 1402016.8

(51) Int. Cl. A61M 16/20; A61M 16/12; A61M 16/06; A61M 16/04; A61M 16/00; A61M 16/08

(52) U.S. Cl. CPC A61M 16/208; A61M 16/0063; A61M 16/0875; A61M 16/06; A61M 16/04; A61M 16/125; A61M 2202/0208

(57) ABSTRACT

A gas-powered, pneumatic ventilator (1) is driven by compressed air from a compressor (2) and its outlet (11) is connected via corrugated tubing (30) to a patient valve (34) and face mask or the like. A source (4) of pure oxygen, such as from an oxygen cylinder (40), is connected via small bore tubing (38) to a gas inlet (37) adjacent the air inlet (33) of the patient valve (34) so that supplementary oxygen is mixed with the air. The patient valve (34) closes and a patient dump valve (16) in the ventilator opens during the exhalation phase to allow oxygen supplied to the patient valve (34) to fill the corrugated tubing (30) from the patient end (32) and displace air in the tubing out of the patient dump valve (16) so that a volume of oxygen is collected in the tubing and is dispensed to the patient during the next inspiratory phase.

This invention relates to pneumatic, gas-powered ventilators of the kind having an inlet arranged for connection to a source of compressed air, an outlet and a mechanism arranged to provide a cyclical air supply for patient ventilation to the outlet via a gas flow path, a length of tubing extending from the outlet to a patient valve unit arranged for connection to a breathing device.

Portable gas-powered, pneumatic ventilators are widely used in both emergency and transport situations. The ventilators can be rugged and simple to operate, which makes them especially suitable for use outside hospitals and by less qualified people, such as paramedics. Such ventilators are usually powered by compressed oxygen, from a cylinder via a reducing valve and most have the ability to entrain air allowing the delivery of between 100% and 50% oxygen. In remote or disaster circumstances, however, the availability of compressed oxygen may be very limited and ideally should be restricted to use with patients requiring supplementary oxygen. Pneumatic ventilators, although designed to be powered by compressed oxygen at between 4 and 6 bars, can also be driven by compressed air with certain associated variation in their calibration. Power to drive an electrical compressor can be obtained from a number of sources apart from the mains, such as a generator or automobile batteries so lack of electrical power is much less of a problem. However, ventilators driven by compressed air can only deliver oxygen to the patient at the same concentration as in air, that is, at 21%. Pneumatic ventilators are available, for example, from Smiths Medical under the PneuPac and VentiPac names (PneuPac and VentiPac are trade marks of Smiths Medical). Conventional arrangements for increasing the concentration of oxygen can be complex or relatively wasteful of oxygen, which can mean that the oxygen source is rapidly exhausted. U.S. Pat. No. 4,898,167 describes a manual bellows ventilator with an oxygen inlet close to the bellows. GB2174608 describes a respirator with an oxygen source connected to an inhalation line. WO2007/008619 describes a respirator with an oxygen source between a gas source and the patient. U.S.2012/0125338 describes a mask system with an oxygen line extending into the mask.

It is an object of the present invention to provide an alternative ventilator and ventilator system.

According to one aspect of the present invention there is provided a ventilator of the above-specified kind, characterised in that the patient valve unit is arranged to enable flow of gas from the tubing to the patient during an inspiratory phase but to prevent flow of gas from the tubing to the patient during an expiratory phase, characterised in that the ventilator includes a ventilator valve connected in the gas flow path that is arranged to be closed during an inspiratory phase and to be open to atmosphere during an expiratory phase, and characterised in that the ventilator includes an oxygen inlet located at or adjacent the patient valve unit and arranged for connection to a source of continuous oxygen flow such that during the expiratory phase oxygen flows along the tubing towards the ventilator as air in the tubing is expelled to atmosphere through the ventilator valve such as to provide a volume of oxygen in the tubing that is delivered to the patient during a subsequent inspiratory phase.

The oxygen inlet is preferably formed as a part of a housing of the patient valve unit. The tubing is preferably corrugated tubing.

According to another aspect of the present invention there is provided a ventilator system including a ventilator according to the above one aspect of the invention, characterised in that the system includes the source of oxygen.

The source of oxygen may include a cylinder of compressed pure oxygen.

According to a further aspect of the present invention there is provided a ventilator system including a ventilator according to the above one aspect of the present invention, characterised in that the system includes the source of compressed air.

The source of compressed air may include an air compressor.

A ventilator system including a ventilator, both according to the present invention, will now be described, by way of example, with reference to the accompanying drawing, in which:

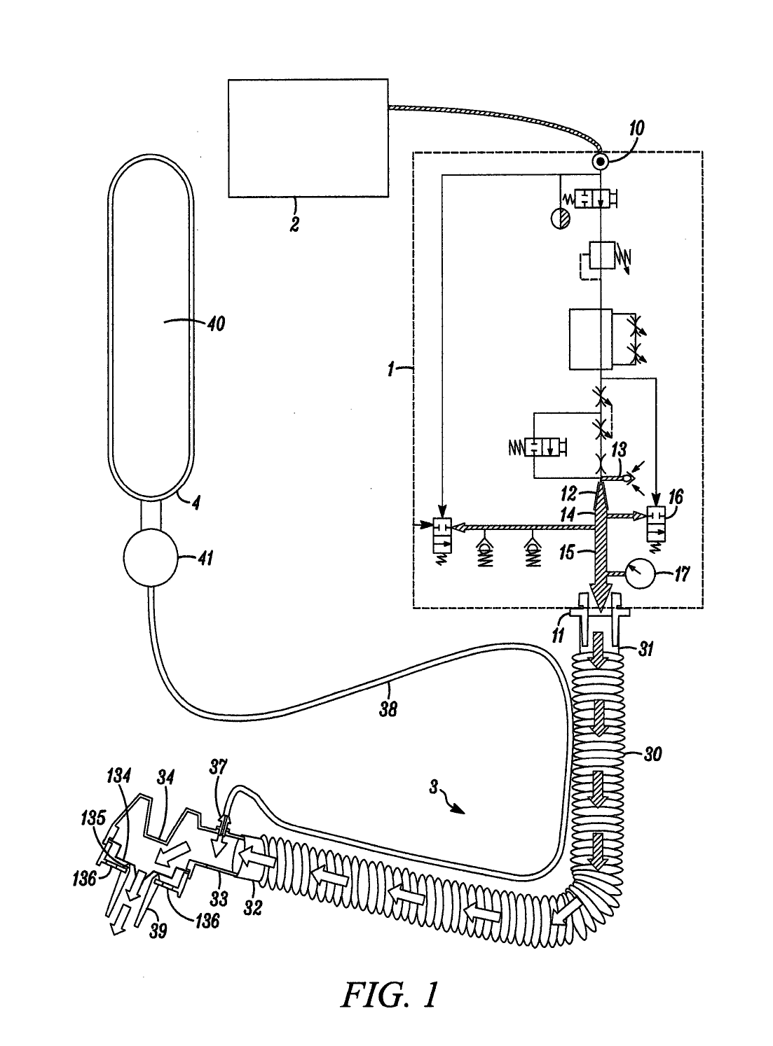

FIG. 1 shows the system schematically during an inspiratory phase some time after start up; and

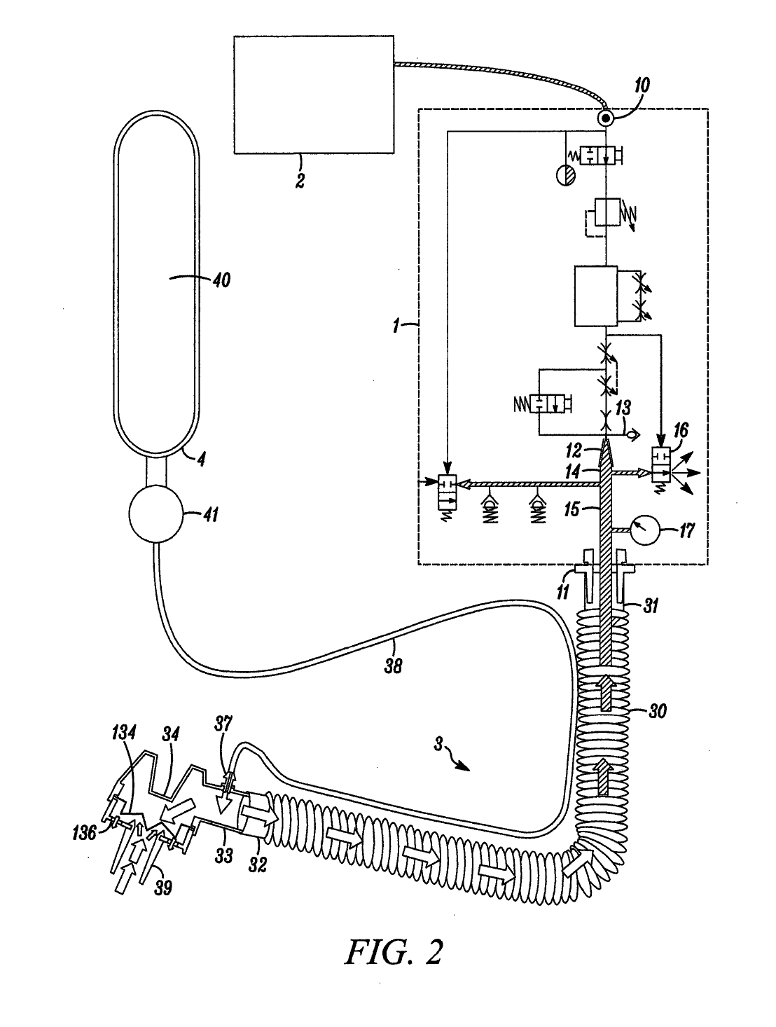

FIG. 2 shows the system during an expiratory phase.

With reference first to FIG. 1, the system comprises a pneumatic, gas-powered ventilator 1, an air compressor 2, a patient breathing circuit 3 and a source of oxygen 4.

The ventilator 1 has an inlet 10 connected with the compressor or some other source of compressed air 2 and an outlet 11 connected with the patient breathing circuit 3. The ventilator 1 is arranged to supply a cyclical output of breathing gas (in this case the breathing gas is air) at a controlled frequency and volume.

The ventilator 1 includes various controls and timing and other valves (details of which are not important for an understanding of the present invention) arranged to produce a flow of air during an inspiratory phase into an air entrainment device 12, which acts to entrain air from a valved inlet 13 so as to produce an increased flow of air at its outlet 14. The outlet 14 of air entrainment device 12 is connected to the ventilator outlet 11 via a gas flow path 15 to which are connected a ventilator or patient dump valve 16 and pressure gauge 17. The patient dump valve 16 is driven by an input upstream of the air entrainment device 12 to be closed during the inspiratory phase but to open during the expiratory phase.

The patient breathing circuit 3 includes a length of corrugated, flexible tubing 30 having one end, its upstream end 31 connected with the outlet 11 of the ventilator 1. The downstream end 32 of the tubing 30 is connected with the inlet 33 of a patient valve housing or unit 34. The patient valve housing 34 includes an outlet 39 for connection to a face mask or other breathing device such as an airway, tracheal tube or the like. The patient valve housing 34 allows gas supplied to its inlet 33 to flow to the outlet 39 and hence to the patient for inspiratory ventilation. The valve 34 includes a duck- bill diaphragm valve 134 that (during inspiration) seals on an annular seat 135 around the outlet 39 to prevent gas escaping via vent outlets 136. During expiration, however, the duckbill in the centre of the diaphragm 134 closes so that pressure can build up in front of the diaphragm sufficient to lift it off the seat 135 to allow the patient to exhale to atmosphere via the vent outlets 136.

The patient valve housing 34 also includes an oxygen inlet 37 in the form of a short, small diameter spigot projecting outwardly on the inlet 33. One end of a length of small bore oxygen tubing 38 is connected with the spigot 37, the opposite end of which is connected to the source of oxygen 4. The source of oxygen 4 includes a cylinder 40 of compressed pure oxygen and a pressure regulator 41. The oxygen source could take other forms where the source provides oxygen at a concentration greater than that in air, for example, it could be provided by a hospital compressed oxygen supply or by an oxygen concentrator or by a chemical oxygen generator. Oxygen is supplied to the patient valve housing 34 at a relatively low rate continuously both during the inspiratory and expiratory phases of the flow. The oxygen inlet is located at or adjacent the patient valve but need not be formed as part of the patient housing since it could instead be provided adjacent the patient valve housing such as on a separate T-piece connected to the machine end of the patient valve housing.

In operation the ventilator 1 is driven by the pressure of air from the compressor 2 supplied to its inlet 10. The patient dump valve 16 is closed to prevent air escaping via this route. The ventilator 1 supplies cyclical ventilation phases of air at 21% oxygen to its outlet 11 and along the tubing 30. Oxygen from the source 4 flows along the tubing 38 to the patient valve housing 34 continuously (both during the inhalation and exhalation phases) where it mixes with the air from the ventilator 1.

Immediately after starting the ventilator 1, during an inspiratory phase, oxygen from the source 4 will flow into the patient valve housing 34 and be driven forwardly, towards the patient by the air flowing out of the tubing 30. At this stage, the air flow along the tubing 30 prevents any significant flow of oxygen rearwardly along the tubing, towards the ventilator 1. FIG. 1 indicates flow during an inspiratory phase but some time after the first cycle.

After a set time, the ventilator 1 switches to an expiratory phase as illustrated in FIG. 2. During this phase the flow of air out of the air entrainment device 12 ceases and the patient dump valve 16 opens to allow gas to escape to atmosphere. The termination of the air flow and the opening of the patient dump valve 16 allows pressure in the tubing 30 and in the patient valve housing 34 to drop sufficiently for the duckbill in the centre of the diaphragm 134 to be closed by pressure on the patient side of the diaphragm and for the diaphragm to be lifted away from the valve seat 135 so that exhaled air from the patient can escape to atmosphere through the openings 136. During this phase, oxygen continues to flow into the patient valve housing 34 from the source 4 and this now flows rearwardly along the tubing 30, pushing at least a part of the air in front of the oxygen column out to atmosphere via the patient dump valve 16 and filling the tubing with oxygen to an extent dependent on the length and cross-sectional area of the tubing and the rate of flow of oxygen from the source 4. Pure oxygen flow is indicated by the outline arrows, air being indicated by the solid arrows within the tubing 30 both in FIG. 1 and FIG. 2. The tubing 30 thereby acts as a reservoir for the oxygen during the expiratory phase. In this way, during the next inspiratory phase the air from the ventilator 1 pushes the column of oxygen collected in the tubing 30 out to the patient via the patient valve housing 34.

In this way the amount of oxygen in the gas supplied to the patient can be increased. It has been found that a supplementary oxygen flow rate of only about 21/min is sufficient to increase oxygen concentration from 21% to 50% at a tidal volume of 600 ml and a frequency of 11 breaths per minute.

The arrangement of the present invention allows for a very simple modification of conventional equipment requiring only a continuous low flow of oxygen, without the need to interrupt flow from the oxygen source and without the need to synchronise flow with patient breathing. The arrangement is also highly efficient since all the oxygen supplied is delivered to the patient providing that flow is adjusted to the volume of the breathing tubing to ensure that it is only air that is vented through the ventilator or patient dump valve during expiration, and not pure oxygen.

The present invention enables useful therapeutic levels of oxygen at above atmospheric levels to be delivered by pneumatic ventilators driven by compressed air. The invention can make best use of oxygen where this is in scarce supply or is expensive.

1-7 (canceled)

8. A pneumatic, gas-powered ventilator having an inlet arranged for connection to a source of compressed air, an outlet and a mechanism arranged to provide a cyclical air supply for patient ventilation to the outlet via a gas flow path, a length of tubing extending from the outlet to a patient valve unit arranged for connection to a breathing device, characterized in that the patient valve unit is arranged to enable flow of gas from the tubing to the patient during an inspiratory phase but to prevent flow of gas from the tubing to the patient during an expiratory phase, characterized in that the ventilator includes a ventilator valve connected in the gas flow path that is arranged to be closed during an inspiratory phase and to be open to atmosphere during an expiratory phase, and characterized in that the ventilator includes an oxygen inlet located at or adjacent the patient valve unit and arranged for connection to a source of continuous oxygen flow such that during the expiratory phase oxygen flows along the tubing towards the ventilator as air in the tubing is expelled to atmosphere through the ventilator valve such as to provide a volume of oxygen in the tubing that is delivered to the patient during a subsequent inspiratory phase.

9. A ventilator according to claim 8, characterized in that the oxygen inlet is formed as a part of a housing of the patient valve unit.

10. A ventilator according to claim 8, characterized in that the tubing is corrugated tubing.

11. A ventilator system including a pneumatic, gas-powered ventilator having an inlet arranged for connection to a source of compressed air, an outlet and a mechanism arranged to provide a cyclical air supply for patient ventilation to the outlet via a gas flow path, a length of tubing extending from the outlet to a patient valve unit arranged for connection to a breathing device, wherein the patient valve unit is arranged to enable flow of gas from the tubing to the patient during an inspiratory phase but to prevent flow of gas from the tubing to the patient during an expiratory phase, wherein the ventilator includes a ventilator valve connected in the gas flow path that is arranged to be closed during an inspiratory phase and to be open to atmosphere during an expiratory phase, and wherein the ventilator includes an oxygen inlet located at or adjacent the patient valve unit and arranged for connection to a source of oxygen having a continuous oxygen flow such that during the expiratory phase oxygen flows along the tubing towards the ventilator as air in the tubing is expelled to atmosphere through the ventilator valve such as to provide a volume of oxygen in the tubing that is delivered to the patient during a subsequent inspiratory phase, wherein the system includes the source of oxygen.

12. A ventilator system according to claim 11 characterized in that the source of oxygen includes a cylinder of compressed pure oxygen.

13. A ventilator system including a pneumatic, gas-powered ventilator having an inlet arranged for connection to a source of compressed air, an outlet and a mechanism arranged to provide a cyclical air supply for patient ventilation to the outlet via a gas flow path, a length of tubing extending from the outlet to a patient valve unit arranged for connection to a breathing device, wherein the patient valve unit is arranged to enable flow of gas from the tubing to the patient during an inspiratory phase but to prevent flow of gas from the tubing to the patient during an expiratory phase, wherein the ventilator includes a ventilator valve connected in the gas flow path that is arranged to be closed during an inspiratory phase and to be open to atmosphere during an expiratory phase, and wherein the ventilator includes an oxygen inlet located at or adjacent the patient valve unit and arranged for connection to a source of oxygen having a continuous oxygen flow such that during the expiratory phase oxygen flows along the tubing towards the ventilator as air in the tubing is expelled to atmosphere through the ventilator valve such as to provide a volume of oxygen in the tubing that is delivered to the patient during a subsequent inspiratory phase, wherein the system includes the source of compressed air.

14. A ventilator system according to claim 13, characterized in that the source of compressed air includes an air compressor.
